(19) United States
(12) Patent Application Publication
(10) Pub. No.: US 2023/0346560 A1
(43) Pub. Date: Nov. 2, 2023

(54) Barrier-forming device for bone regeneration

(71) Applicant: Georges Khoury, Paris (FR)

(72) Inventor: Georges Khoury, Paris (FR)

(21) Appl. No.: 18/306,523

(22) Filed: Apr. 25, 2023

(30) Foreign Application Priority Data
May 2, 2022 (FR) 22 04158

(51) Int. Cl. A61F 2/28

(52) U.S. Cl. CPC A61F 2/28; A61L 2430/02; A61B 2017/00004

(57) ABSTRACT

A device for bone regeneration intended to form a barrier for protecting a space above a surface of a bone, for example a tooth, to allow, after insertion of a biomaterial, which, while mineralising, will form new bone above existing bone, includes a self-supporting barrier-forming element in the shape of a dome (1) or a shell, and means for attaching the barrier-forming element above the bone surface in order thus to form a volume where the biomaterial can be arranged to mineralise and form new bone grafting itself onto the previously present bone, is characterised in that the barrier-forming element in the shape of a dome (1) or shell is made of a material, specifically a non-resorbing material, such that the barrier-forming element is radiotransparent

The present invention relates to a device intended to form a barrier in the shape of a dome or shell, specifically in the field of bone regeneration, which allows a volume to be created and protected above the surface of a remaining bone in order to arrange therein a biomaterial element which, while mineralising under the protection of the dome vis-à-vis other tissues, will form new bone in the volume protected by the dome while aggregating on the surface of the remaining bone

Already in the prior art, for example FR2713090A1, a shell of this type in the shape of a semicylindrical tube made of titanium, which is held above an osseous surface with which it is desired to join together osseous tissue by regeneration, by two edge seals or a peripheral seal acting as a means of holding the shell away from the surface on which it is desired to regenerate the bone, the space inside the shell being protected from gingival tissue

The present invention aims to improve devices of the prior art by making available a barrier-forming device of the type named above, which allows even further improvement of the precision of the volume of regenerated bone, in order thus to obtain a new bone corresponding as precisely as possible to what the surgeon wishes to obtain initially.

According to a first aspect of the invention, a device for bone regeneration intended to form a barrier for protecting a space above a surface of a bone, for example the jaws, to allow, after insertion of a biomaterial, which, while mineralising, will form new bone above existing bone, comprising a self-supporting barrier-forming element in the shape of a dome or a shell and means for attaching the barrier-forming element above the bone surface in order thus to form a volume where the biomaterial can be arranged to mineralise and form new bone grafting itself onto the previously present bone, is characterised in that the barrier-forming element in the shape of a dome or shell is made of a material, specifically a non-resorbing material, such that the barrier-forming element is radiotransparent, i.e. specifically allows through X-rays with a transmission coefficient of at least 60%, preferably at least 70%, preferably 80%, even more preferably 90%, specifically substantially 100%.

By thus providing a material which is sufficiently transparent to X-rays, the surgeon can see, with sufficient precision, the inner volume formed between the barrier element and the bone surface to which it is desired to join the bone material, and thus precisely shape this volume, in order to adapt it as far as possible to the shape which it intends to give to the final implant.

Preferably, and according to another aspect of the invention which per se constitutes an invention, but which can advantageously be implemented in combination with the first aspect, the barrier element is self-supporting but can be deformed elastically, specifically by compression and/or flexion.

Preferably, the device for bone regeneration forms a barrier for protecting, hermetically, the space above a surface of a bone, for example jaws.

In particular, the barrier element can be deformed elastically by compression, this compression capacity allowing a better osseous formation to be obtained.

It is thus possible to shape the volume in which it is desired that the osseous material is regenerated as precisely as possible, and thus obtain an implant with the shape as close as possible to that which a surgeon wishes to obtain at the start of their intervention. Moreover, the barrier element can easily be produced by 3D printing.

In particular, the biomaterial from which the barrier element is formed has a Young's modulus of between 2 and 20 GPa, specifically between 2 and 15 GPa, more particularly between 2 and 5 GPa, specifically between 3 and 4 GPa.

According to a preferred embodiment of the embodiment, the material has a flexural strength of between 100 and 200 MPa, specifically between 150 and 190 MPa, specifically substantially equal to 180 MPa.

According to a preferred embodiment of the embodiment, the material of the barrier element has a tensile strength of between 50 and 120 MPa, specifically between 80 and 100 MPa.

The above values for resistance and Young's modulus are given at ambient temperature, i.e. between 20 and 25° C., specifically 23° C., and at atmospheric pressure.

According to a particularly preferred embodiment, the material of the non-resorbing barrier element is a polyaryletherketone (PAEK), specifically a polyetheretherketone (PEEK) or a polyetherketoneketone (PEKK).

Other examples of suitable materials include polylactic acid (PLA) and polyglycolic acid (PGA) which are resorbing.

According to a preferred embodiment of the embodiment, the barrier-forming element comprises at least one runner in the shape of a slit, specifically two runners in the shape of slits, preferably four runners in the shape of lateral slits which extend in the direction perpendicular to the opening opposite the top of the barrier in the shape of a dome or shell and one or some corresponding respective osteosynthesis screw(s), arranged in a respective slit runner allowing the barrier-forming element to be attached around the surface of the bone to be regenerated, the movement of the screws in the runners allowing the volume protected by the barrier-forming element to be controlled and the pressure of the barrier-forming element on the biomaterial to be maintained, the obtained pressure due to the possible compression deformation of the barrier-forming element.

Preferably, the device for bone regeneration forms a barrier for protecting, hermetically, the space above a surface of a bone, for example jaws.

By way of example, an embodiment of the invention is described with reference to the drawings, in which:

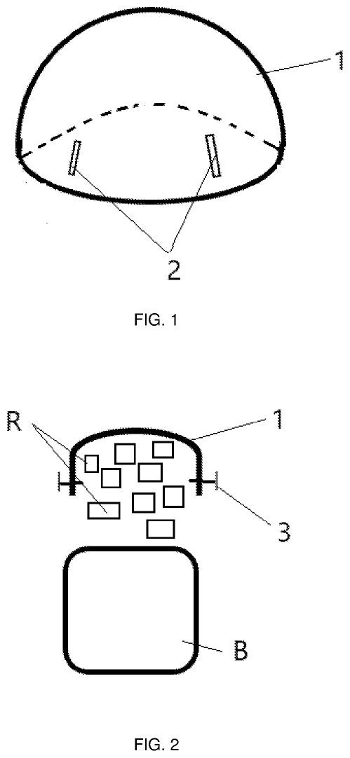

FIG. 1 is a perspective view of a barrier element according to the invention in the shape of a dome;

FIG. 2 is a schematic cross-section of the barrier-forming element of FIG. 1 arranged above a bone before its installation; and

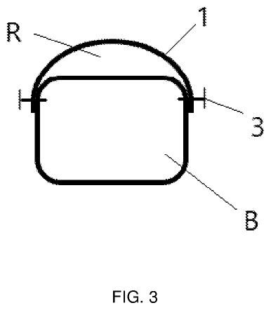

FIG. 3 is a view similar to that of FIG. 2 in the installed position of the barrier.

In FIG. 1 there is shown a dome 1 made of PEEK having a thickness of 0.5 mm, this thickness being able specifically to be between 0.1 mm and 1 mm. Four runner slits 2 extend substantially vertically, near the opening of the dome, formed opposite the top of the dome. Each of the runner slits 2 receives a respective screw 3 which can slide in its respective slit.

As shown in FIGS. 2 and 3, dome 1 is intended to cover a surface of a bone B by leaving there a volume protected from other soft tissues, such as gingiva or skin, or the like, in order to be able to insert there osseous biomaterial which, while mineralising under the protection of the dome vis-à-vis other tissues, will form new bone in the volume protected by the dome while aggregating onto the surface of bone B.

Once the inner volume of the dome is filled with biomaterial R intended to be developed, the surgeon can apply a pressure to the dome to compress the biomaterial located therein and, using screws, fix the dome in final position in which it retains a shape (FIG. 3) under pressure deformed in respect of its initial shape (FIG. 2).

On the other hand, the surgeon can likewise easily see the shape of the inner space of the X-ray dome and adapt same to the shape of the implant intended to be achieved, while being able, due to the screw attachment and the deformable nature of the dome wall, to add or remove biomaterial intended to form regenerated bone.

As seen in FIG. 1 specifically, the wall of the dome is preferably solid, without any opening crossing it except for very fine slits 2, specifically of the order of 0.5 to 3 mm in size, with a length of the order of 2 to 15 mm.

Preferably, the height of the dome, i.e. the distance perpendicular to its opening up to the top, is a function of the need for bone gain, in general of the order of 3 to 15 mm, in the dental field, whereas this length can be greater, in the order of several centimetres, in the orthopaedic or maxillofacial field.

What is claimed is:

1. A device for bone regeneration intended to form a barrier for protecting a space above a surface of a bone (B), for example a tooth, to allow, after insertion of a biomaterial, which, while mineralising, will form new bone above existing bone, comprising a self-supporting barrier-forming element in the shape of a dome (1) or a shell and means for attaching the barrier-forming element above the bone surface in order thus to form a volume where the biomaterial can be arranged to mineralise and form new bone grafting itself onto the previously present bone, characterised in that the barrier-forming element in the shape of a dome (1) or shell is made of a material, specifically a non-resorbing material, such that the barrier-forming element is radiotransparent, i.e. specifically allows through X-rays with a transmission coefficient of at least 60%, preferably at least 70%, preferably 80%, even more preferably 90%, specifically substantially 100%.

2. The device according to claim 1, characterised in that the barrier element (1) is self-supporting but can be deformed elastically, specifically by compression and/or flexion.

3. The device according to claim 2, characterised in that the barrier element (1) is self-supporting but can be deformed elastically by compression and/or flexion.

4. The device according to claim 1, characterised in that the material from which the barrier element is made has a Young's modulus of between 2 and 20 GPa, specifically between 2 and 15 GPa, more particularly between 2 and 5 GPa, specifically between 3 and 4 GPa.

5. The device according to claim 1, characterised in that the material of the barrier element has a flexural strength of between 100 and 200 MPa, specifically between 150 and 190 MPa, specifically substantially equal to 180 MPa.

6. The device according to claim 1, characterised in that the material of the barrier element has a tensile strength of between 50 and 120 MPa, specifically between 80 and 100 MPa.

7. The device according to claim 1, characterised in that the material of the barrier element is non-resorbing.

8. The device according to claim 7, characterised in that the non-resorbing material is a polyaryletherketone (PAEK).

9. The device according to claim 8, characterised in that the barrier element is a PAEK in the form of a polyetheretherketone (PEEK) or a polyetherketoneketone (PEKK).

10. The device according to claim 1, characterised in that the material of the barrier element is resorbing.

11. The device according to claim 10, characterised in that the resorbing material is polylactic acid (PLA) and/or polyglycolic acid (PGA).

12. The device according to claim 1, characterised in that the barrier element (1) comprises at least one runner (2) in the shape of a slit, specifically two runners in the shape of slits, preferably four runners in the shape of lateral slits which extend in the direction perpendicular to the opening opposite the top of the barrier in the shape of a dome or shell and one or some corresponding respective osteosynthesis screw(s) (3), arranged in a respective slit runner allowing the barrier-forming element to be attached around the surface of bone to be regenerated, the movement of the screws in the runners allowing the volume protected by the barrier-forming element to be controlled, due to the possible deformation of the barrier-forming element.

13. The device according to claim 1, characterised in that the device for bone regeneration forms a barrier for protecting, hermetically, the space above a surface of a bone, for example jaws.
